(19) United States
(12) Patent Application Publication
(10) Pub. No.: US 2004/0180003 A1
(43) Pub. Date: Sep. 16, 2004

(54) Methods of screening for caloric restriction mimetics and reproducing effects of caloric restriction

(76) Inventors: Stephen R. Spindler, Riverside, CA (US); Joseph M. Dhabi, Riverside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/387,743

(22) Filed: Mar. 12, 2003

(51) Int. Cl. A61K 49/00

(52) U.S. Cl. 424/9.1

(57) ABSTRACT

A method for searching for a compound that mimics the effects induced by a caloric restriction (CR) program. The method comprises administering a CR diet program to a first group of mammals for a predetermined amount of time and administering a dosage of at least one compound to a second group of mammals for a term which is less than or equal to the predetermined amount of time. The method further comprises assessing changes in gene expression levels, levels of nucleic acids, proteins, or protein activity levels and determining whether the compound mimics effects induced by the CR diet program.

### BACKGROUND

1. Field

Many aspects of this disclosure relate to methods of screening for caloric restriction (CR) mimetics and reproducing at least some of the effects induced by CR. For example, methods of identifying compounds that reproduce at least some of the effects induced by CR and identifying compounds that delay the onset of age related diseases or extend longevity are disclosed.

2. Discussion of Related Art

A major goal of pharmaceutical research has been to discover ways to reduce morbidity and delay mortality. However, there are presently no authentic longevity pharmaceuticals. One reason for that is that no assay has existed for identifying such drugs. Several decades ago it was discovered that a decrease in caloric intake, termed caloric restriction (CR), can significantly and persistently extend healthy life in animals; see for example, Weindruch, et. al., The Retardation of Aging and Disease by Dietary Restriction, (Charles C. Thomas, Springfield, Ill.), 1988. CR remains the only reliable intervention capable of consistently extending lifespan and reducing the incidence and severity of many age-related diseases, including cancer, diabetes and cardiovascular diseases. Additionally, physiological biomarkers linked to lifespan extension in rodents (e.g., mice, rabbits, shrews, and squirrels) and monkeys that have been subjected to CR have been shown to associate with enhanced lifespan in humans; see for examples, Weyer, et. al., Energy metabolism after 2 years of energy restriction: the biosphere 2 experiment , Am. J. Clin. Nutr. 72, 946-953, 2000, and Roth, et. al., Biomarkers of caloric restriction may predict longevity in humans, Science 297, 811, 2002. A study by Walford et. al. indicated that healthy nonobese humans on CR diets show physiologic, hematologic, hormonal, and biochemical changes resembling those of rodents and monkeys on such CR diets. See Walford, et. al., Calorie Restriction in Biosphere 2: Alternations in Physiologic, Hematologic, Hormonal, and Biochemical Parameters in Humans Restricted for a 2 -Year Period, J. Gerontol.: Biol. Sci. 57A, 211-224, 2002. These preliminary findings suggest that the anti-aging effects of CR may be universal among all species. The molecular-genetic processes that lead to lifespan extension and reduce disease incident in animals may extend lifespan and reduce disease incidence in humans.

Historically, the only accepted assay for evaluating compounds for their effects on aging and the development of age-related diseases has been lifespan studies. However, this method has distinct limitations. Even a “short-lived” mammal like a mouse lives 40 months. Use of a shorter-lived, enfeebled rodent strain introduces confounds into the study. A cohort of at least 60 rodents is required to have the statistical power to reliably detect a 10% change in longevity. Thus, a large-scale CR mimetic screening is impractical using this standard. For more than 25 years, scientists have been searching for biomarkers that would make it possible to detect the development of age-related diseases and the underlying rate of aging over short periods of time. For the most part, these efforts have not met with success.

### SUMMARY

Even though CR brings many benefits to animals and humans, it is not likely that many will avail themselves of a CR lifestyle. Additionally, few are able to maintain weight loss. The identification and development of CR mimetic compounds or drugs are thus desirable. CR mimetic compounds or drugs are compounds capable of mimicking at least some of the anti-aging, anti-disease effects, and other beneficial effects of CR without a substantial reduction in dietary calorie intake or without reducing the subject's weight below a normal weight.

Certain exemplary embodiments of the present invention allow screening and/or evaluation of at least one compound that mimics or reproduces the effects or some of the effects induced by CR in mammals, for example, mice. In one embodiment, the effectiveness of several compounds (e.g., Metformin, Glipizide, Rosiglitazone, and Soy Isoflavones as well as combinations thereof) are identified and evaluated as CR mimetics because they reproduce at least some of the effects induced by CR. The effects induced by CR and each of the compounds, alone, or in combination, in organs (e.g., livers, hearts, and brains) of mice are evaluated. In one embodiment, gene-expression profiles of mice subjected to CR and mice subjected to the administration of the compounds are evaluated and compared. In other embodiments, a compound or compounds are screened for their ability to inhibit or retard the aging process in mammals.

One embodiment describes a method for searching for a compound that mimics at least some of the effects induced by a CR program. The method comprises administering a CR diet program to a first group of mammals for a predetermined amount of time and administering a dosage of at least one compound to a second group of mammals for a term which is less than or equal to the predetermined amount of time. The method further comprises assessing changes in gene expression levels, levels of nucleic acids, proteins, or protein activity levels and determining whether the agent mimics the effects induced by the CR diet program.

Another embodiment describes a method of reproducing at least one effect in mammals that have been subjected to long-term caloric restriction (LT-CR). The method comprises administering a LT-CR diet program to a first group of mammals for a first duration of time and administering at least one compound to a second group of mammals for a second duration of time. The second duration of time is substantially shorter than the first duration of time. The first group of mammals and the second group of mammals are similar, for example, both are groups of mice. Control data from an administering of a control diet program is obtained. Effects of the LT-CR diet program and the compound are determined by comparing data obtained from the first group of mammals and the second group of mammals to the control data. Effects between the LT-CR diet program and the compound are compared to determine whether the compound reproduces at least one effect caused by the LT-CR.

Another embodiment describes a method of identifying a compound that reproduces effects of a CR. The method comprises administering an effective dosage of a compound to a first group of mammals for a duration of time; administering a CR diet program to a second group of mammals; and obtaining control data from an administering of a control diet program. The first group of mammals and the second group of mammals are similar, for example, both are groups of mice. The method further comprises analyzing changes in gene expression levels, levels of nucleic acids, protein, or protein activity levels, in each of the first group of mammals and the second group of mammals. The compound is identified as one that reproduces changes induced by CR when the compound produces analyzed changes in the first group of mammals wherein at least about 1% or one or more gene changes of the analyzed changes are a subset of the changes induced by the CR. In one embodiment, the changes in gene expression levels, levels of nucleic acids, protein, or protein activity levels, in each of the first group of mammals and the second group of mammals are compared to the control data to identify and compare the changes.

Another embodiment describes a method for searching for a compound. The method comprises administering a ST-CR diet program to a first group of mammals for a predetermined amount of time and administering a dosage of at least one compound to a second group of mammals, for a term which is less than or equal to the predetermined amount of time. The method further comprises assessing changes in gene expression levels, levels of nucleic acids, proteins, or protein activity levels and determining the compound's mimetic effects induced by the ST-CR diet program.

Another embodiment describes a method of extending longevity (or increasing maximum life span) for a mammal that is otherwise healthy. The method comprises administering an effective dosage of at least one of Metformin, Glipizide, Rosiglitazone, and Soy Isoflavones (or combinations thereof) to the mammal for an effective amount of time.

Another embodiment disclosed a method of reproducing effects of CR comprising administering an effective dosage of at least one of Metformin, Glipizide, Rosiglitazone, and Soy Isoflavones to a mammal for an effective amount of time.

In other embodiments, the biological age or metabolic state of an organism (e.g., a mammal) may be assessed by determining the gene expression level of one or more of the genes listed in Tables 3-7. These and other features and advantages of embodiments of the present invention will be more readily apparent from the detailed description of the embodiments, set forth below, taken in conjunction with the accompanying drawings.

### BRIEF DESCRIPTION OF THE DRAWINGS

The present invention is illustrated by way of example and not limitation in the figures of the accompanying drawings, in which like references indicate similar elements and in which:

FIG. 1 illustrates an exemplary dietary regimen scheme that various test groups are subjected to;

FIG. 2 illustrates an analysis of gene expression changes in mouse liver following 8 weeks of treatment with various compounds according to some embodiments;

FIG. 3 illustrates a Venn diagram analysis;

Table 1 illustrates 8 various treatments (with exemplary dosage of the compounds) that can be administered to a test group such as mice;

Table 2 illustrates percentage of compound-specific or drug-specific effects and overlap between the effects of CR and those of each of the treatments used;

Table 3 illustrates effects of Metformin and CR on hepatic gene expression;

Table 4 illustrates effects of Glipizide and CR on hepatic gene expression;

Table 5 illustrates effects of Glipizide and Metformin and CR on hepatic gene expression;

Table 6 illustrates effects of Rosiglitazone and CR on hepatic gene expression;

Table 7 illustrates effects of Soy Isoflavones and CR on hepatic gene expression;

Table 8 illustrates genes with gene expression that are altered in the opposite direction by LT-CR and the compounds/drugs being tested; and

Table 9 illustrates the percentage of CR effects reproduced by different compounds.

The features of the described embodiments are specifically set forth in the appended claims. However, the embodiments are best understood by referring to the following description and accompanying drawings, in which similar parts are identified by like reference numerals.

### DETAILED DESCRIPTION

Exemplary embodiments are described with reference to specific configurations and techniques. Certain embodiments of the present invention pertain to methods of screening for CR mimetics and reproducing the effects induced by CR. Methods of identifying compounds that reproduce the effects induced by CR, identifying compounds that delay the onset of age related diseases or extend longevity, and extending longevity in mammals are disclosed. In the following description, for purposes of explanation, numerous specific details are set forth in order to provide a thorough understanding of the exemplary embodiments of the present invention. It will be evident, however, to one skilled in the art, that these embodiments may be practiced without these specific details. In other instances, specific structures and methods have not been described so as not to obscure the present invention. The following description and drawings are illustrative of the invention and are not to be construed as limiting the invention.

Currently, CR when started either early in life or in middle-age, represents the best established paradigm of aging retardation in mammals. See for example, Weindruch, et. al., The Retardation of Aging and Disease by Dietary Restriction, C. C. Thomas, Springfield, Ill., 1988. The effects of CR on age-related parameters are broad. CR increases maximum lifespan, reduces and delays the onset of age related diseases, reduces and delays spontaneous and induced carcinogenesis, suppresses autoimmunity associated with aging, and reduces the incidence of several age-induced diseases (Weindruch, supra, 1988). For example, CR delays the onset of kidney disease, cancer, autoimmune disease, and diabetes. CR reduces neuronal loss with age in mouse models of neurodegenerative disorders, including Parkinson's disease and Alzheimer's disease. CR also prevents declines in psychomotor and spatial memory tasks with age and dendritic spine loss. CR also enhances the brain's plasticity and repair.

Even though CR brings many beneficial effects to animals and humans, it is not likely that many will avail themselves of a CR lifestyle. As is known, it is difficult for any animal or human to maintain a diet program similar to a CR diet program. There is thus a need to identify, evaluate, and develop CR mimetic compounds or drugs that are capable of mimicking at least some of the anti-aging, anti-disease effects, and other beneficial effects of CR without the reduction of dietary calorie intake as required by CR diet programs.

In one embodiment, a mammalian sample group is chosen. In one case, the mammalian sample group is a group of mice, or laboratory mice. The mice are divided into groups, each of which will undergo a different treatment. One group of mice is subjected to a CR diet program (reduced number of calories in the diet). Another group of mice can be a control group, which is subjected to a control (normal number of calories) diet program. Other groups of mice can be used for testing compounds (e.g., pharmaceutical compounds or agents) to determine whether these compounds will reproduce the effects (or at least some of the effects) of CR. The effects caused by different treatments to mice in these groups are then compared to the control group and/or to each other. Comparing the effects of CR and the various compounds on the mice will allow determination or identification of the CR mimetic compounds. It will be recognized that the various embodiments described herein can be used with non-mammal organisms such as insects, nematodes, yeast, bacteria, and other organisms. Thus, the screening techniques may be performed in these non-mammal organisms and then candidate drugs, discovered in those organisms, can be tested in mammals (e.g., humans).

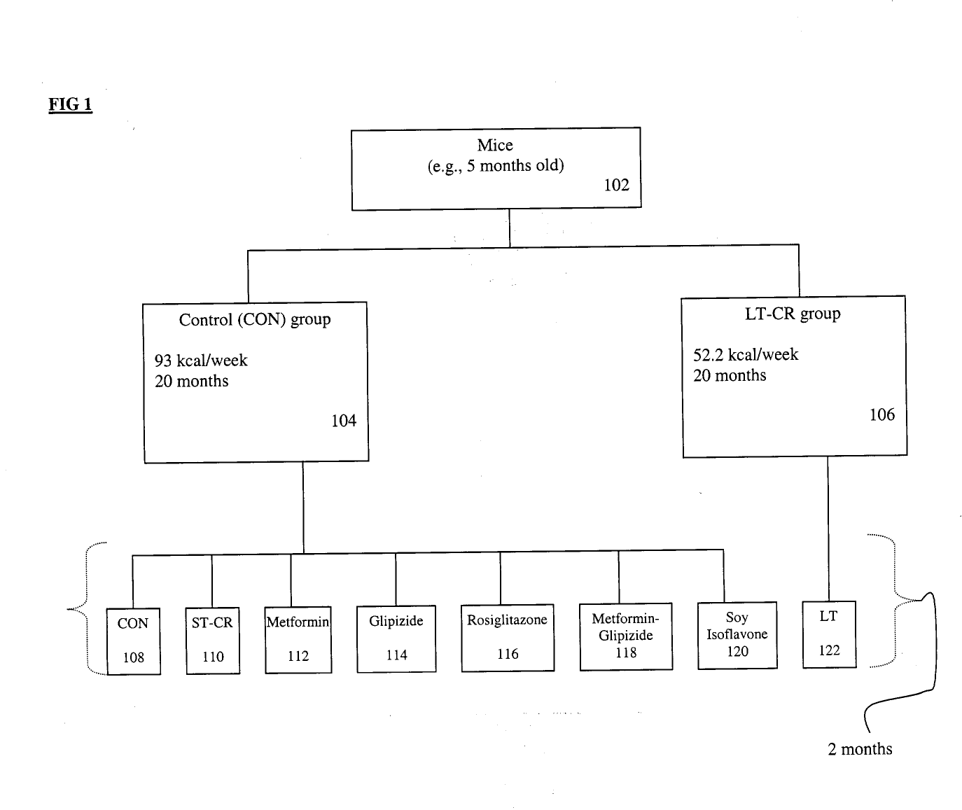

FIG. 1 illustrates an exemplary scheme 100 of the various dietary regimens or programs and compound administration programs for mammalian samples. In one embodiment, the mammalian samples are mice. One-month-old male mice of the long-lived strain C57B16×C3H F1 were purchased from Harlan (Indianapolis, Ind.). Mice were housed in groups of four per cage and fed a non-purified diet, PMI Nutrition International Product # 5001 (Purina Mills, Richmond, Ind.). In one embodiment, at five months of age, the mice were individually housed. In one embodiment, at five months, the mice are subjected to various diet or treatment programs. As illustrated in FIG. 1, the five-month old mice as shown in box 102 were randomly assigned to one of two groups, a control (CON) group 104, and a long-term CR (LT-CR) group 106. In one embodiment, each mouse in the CON group 104 was fed 93 kcal per week of the purified control diet (AIN-93M, Diet No. F05312, BIO-SERV). In one embodiment, each mouse in the LT-CR group 106 was fed 52.2 kcal per week of a purified CR diet (AIN-93M 40% Restricted, Diet No. F05314, BIO-SERV). In one embodiment, each mouse in the LT-CR mice 106 consumed approximately 40% fewer calories than each mouse in the CON group 104. The CR diet was enriched in protein, vitamins, and minerals so that the CR mice consumed approximately the same amount of these nutrients per gram body weight as the control mice. Mice had free access to acidified tap water. No signs of pathology were detected in any of the animals used. All animal use protocols were approved by an institutional animal use committee.

In one embodiment, at 20 months of age, mice in the LT-CR group 106 continued to be fed with the CR diet for another two months (eight weeks). The mice in the CON group 104 were divided into various groups subjected to various test compounds and in one embodiment, the test compounds are gluco-regulatory compounds. In one embodiment, the mice in the CON group 104 were randomly assigned to seven experimental groups, a CON group 108, a short-term CR (ST-CR) group 110, a Metformin group 112, a Glipizide group 114, a Rosiglitazone group 116, a Metformin-Glipizide combination group 118, and a Soy Isoflavone group 120. Metformin, Glipizide, Rosiglitazone, and Soy Isoflavones are some of the test compounds that can be used. Metformin, Glipizide, and Rosiglitazone are examples of glucoregulatory compounds. Each mouse in the CON group 108 continued to be fed 93 kcal per week of control diet alone for eight weeks. Each mouse in the ST-CR group 110 was fed 77 kcal per week of CR diet for two weeks, followed by 52.2 kcal per week of CR diet for six weeks. The mice in the other five groups were fed the control diet containing one drug or a combination of two drugs for a total of eight weeks. The drug or compound administration can be shorter than eight weeks, for example, between about 1 day to about 8 weeks. In one embodiment, each mouse in the Metformin group 112 was fed the 93 kcal per week control diet plus 2100 mg of Metformin in 1 kg of the control diet; each mouse in the Glipizide group 114 was fed the 93 kcal per week control diet plus 1050 mg of Glipizide in 1 kg of the control diet; each mouse in the Rosiglitazone group 116 was fed the 93 kcal per week control diet plus 80 mg of Rosiglitazone in 1 kg of the control diet; each mouse in the Metformin-Glipizide combination group 118 was fed the 93 kcal per week control diet plus 1050 mg of Metformin and 525 mg of Glipizide in 1 kg of the control diet; and, each mouse in the Soy Isoflavone group 120 was fed with the 93 kcal per week control diet having 0.25% (by weight) Soy Isoflavones in the control diet.

The amounts of the drugs or the compounds such as Metformin, Glipizide, Rosiglitazone, and Soy Isoflavones, to be administered to the mice can vary depending on the types of compounds and/or their concentrations. In one embodiment, dosages for Metformin may be approximately between 0.2 mg and 2.0 gm of Metformin per kg body weight per day. Dosages for Glipizide may be approximately between 1.05×10⁻3 mg and 105 mg of Glipizide per kg body weight per day. Dosages for Rosiglitazone may be approximately between 8.0×10⁻4 mg and 8.0 mg of Rosiglitazone per kg body weight per day. The dosages for the combination of Metformin and Glipizide may be approximately between 0.1 mg and 1.0 gm per kg body weight per day of Metformin plus approximately between 0 mg and 52.5 mg of Glipizide per kg body weight per day. The dosages for Soy Isoflavones may be approximately between 0.025-2.5% of daily diet (by weight) of Soy Isoflavones in the control diet.

Metformin was obtained from Sigma, St. Louis, Mo.; Glipizide was also obtained from Sigma; Rosiglitazone (known as Avandia), was obtained from SmithKline Beecham; and Soy Isoflavone extract was NOVASOY 400, obtained from Life Extension Foundation. These compounds were mixed with the powered control diet and cold-pressed into one-gram pellets by the diet supplier (BIO-SERV).

Mice were killed at 22 months of age. They were fasted for 48 hours and killed by cervical dislocation. The organs were removed rapidly, placed in plastic screw-cap tubes, and flash frozen in liquid nitrogen. The tissues were stored in liquid nitrogen.

In one embodiment, mice in the LT-CR group 106 are subjected to the CR diet for a duration of time that is longer or substantially longer than mice in the ST-CR group 110, for example, 5 weeks to 40 months longer. Similarly, mice in the LT-CR group 106 are subjected to the CR diet for a duration of time that is longer or substantially longer (e.g., 5 weeks to 40 months longer) than mice in the drug groups, such as the Metformin group 112, the Glipizide group 114, the Rosiglitazone group 116, the Metformin-Glipizide combination group 118, and the Soy Isoflavone group 120. In some embodiments, mice in the LT-CR group 106 are subjected to the CR diet to about the end of their life.

It is to be noted that other compounds can be chosen in addition to or in place of the compounds (e.g., Metformin, Glipizide, and Rosiglitazone) listed above. In some embodiments, glucoregulatory compounds such as Metformin, Glipizide, and Rosiglitazone, alone and in combination, were tested. Glucoregulatory agents are chosen because CR produces a marked reduction in blood insulin levels (˜50%), lowers blood glucose levels (˜15%) and enhances insulin sensitivity in tissues. These same effects are often produced by glucoregulatory pharmaceuticals. Compounds known to lower circulating glucose and insulin levels are promising candidate CR mimetics. Thus, other test compounds that are glucoregulatory agents can be used in the embodiments of the present invention without deviating from the scope of the disclosure. In addition, small molecule cancer chemopreventatives (e.g., Soy Isoflavones) can also be used in addition to the test compounds listed in FIG. 1 to screen for a CR mimetic compound(s).

It is also to be noted that control data can be obtained from a prior study, the results of which are recorded as opposed to a control group of mice subjected to a control diet program concurrently with the test groups of mice as illustrated in FIG. 1. Thus, the control data may be obtained from an administering of a control diet program which was previously performed. This control data may be obtained once and stored for recall in later screening studies for comparison against the results in the later screening studies. Similarly, gene expression levels from LT-CR or ST-CR (or other types of measurements such as changes in protein levels, changes in protein activity levels, changes in carbohydrate or lipid levels, changes in nucleic acid levels, changes in rate of protein or nucleic acid synthesis, changes in protein or nucleic acid stability, changes in protein or nucleic acid accumulation levels, changes in protein or nucleic acid degradation rate, and changes in protein or nucleic acid structure or function) may be evaluated and recorded once for recall in later screening studies for comparison against the results in the later screening studies. Of course, it is typically desirable to have the prior stored studies have a similar (if not identical) set of genes (or other parameters such as proteins) relative to the genes (or other parameters) in the later screening studies in order to perform a comparison against a similar set of genes or other parameters.

Additionally, a compound can be evaluated or determined to see whether it will reproduce the effects of CR or mimic CR by being fed to the mice in a scheme similar to that illustrated in FIG. 1.

The isolated organs or tissues can be used to perform many different types of analysis that allow for determination of effects of each of the different treatments. The effects include at least one of changes in gene expression levels (e.g., mRNA levels), changes in protein levels, changes in protein activity levels, changes in carbohydrate or lipid levels, changes in nucleic acid levels, changes in rate of protein or nucleic acid synthesis, changes in protein or nucleic acid stability, changes in protein or nucleic acid accumulation levels, changes in protein or nucleic acid degradation rate, and changes in protein or nucleic acid structure or function, to name a few. Some embodiments focus on the determination of changes in gene expression levels. It is to be noted that the exemplary methods discussed are not limited only to analyzing genes expressions that are affected by CR or CR mimetics but are also to include changes in physiological biomarkers such as changes in protein levels, changes in protein activity, changes in levels of nucleic acids, changes in carbohydrate levels, changes in lipid levels, changes in rate of protein or nucleic acid synthesis, changes in protein or nucleic acid stability, changes in protein or nucleic acid accumulation levels, changes in protein or nucleic acid degradation rate, and changes in protein or nucleic acid structure or function, and the like.

In one embodiment, mRNA levels of specific genes or nucleic acid sequences in the different groups of the mice were measured in various organs of the mice. In one embodiment, total liver RNA was isolated from frozen tissue fragments by Tekmar Tissuemizer (Tekmar Co., Cincinnati, Ohio) homogenization in TRI Reagent (Molecular Research Center, Inc., Cincinnati, Ohio) as described by the supplier. mRNA levels were measured using the Affymetrix U74v2A high-density oligonucleotide arrays according to the standard Affymetrix protocol (Affymetrix, Santa Clara, Calif.). Briefly, cDNA was prepared from total RNA from each animal's organ using Superscript Choice System with a primer containing oligo(dT) and the T7 RNA polymerase promoter sequence. Biotinylated cRNA was synthesized from purified cDNA using the Enzo BioArray High Yield RNA Transcript Labeling Kit (Enzo Biochem). cRNA was purified using RNeasy mini columns (Qiagen, Chatsworth, Calif.). An equal amount of cRNA from each animal was separately hybridized to U74v2A high-density oligonucleotide arrays. The arrays were hybridized for 16 hours at 45° C. After hybridization, arrays were washed, stained with streptavidin-phycoerythrin, and scanned using a Hewlett-Packard GeneArray Scanner. Image analysis and data quantification were performed using the Affymetrix GeneChip analysis suite v5.0.

In one embodiment, image analysis and data quantification were performed using Affymetrix Microarray Suite 5.0. The U74vA array contains targets for more than 12,422 mouse genes and expressed sequence tags (ESTs). Each gene or EST is represented on the array by 20 perfectly matched (PM) oligonucleotides and 20 mismatched (MM) control probes that contain a single central-base mismatch. All arrays were scaled to a target intensity of 2500 . The signal intensities of PM and MM were used to calculate a discrimination score, R, which is equal to (PM−MM)/(PM+MM). A detection algorithm utilizes R to generate a detection p-value and assign a Present, Marginal or Absent call using Wilcoxon's signed rank test. Details of this method can be found in Wilcoxon F. Individual Comparisons by Ranking Methods, Biometrics 1, 80-83, 1945 and Affymetrix, I. New Statistical Algorithms for Monitoring Gene Expression on GeneChip Probe Arrays, Technical Notes 1, Part No. 701097 Rev. 1, 2001. Only genes that were “present” in at least 75% of all arrays in an experimental group were considered for further analysis. In addition, genes with signal intensity lower than the median array signal intensity in any of all the arrays were eliminated from the analysis. These selection criteria reduced the raw data from 12,422 genes to 3505 genes that were considered for further analysis. The use of these microarrays allows for rapid gene expression profiling between the groups of test subjects allowing for rapid screening of possible compounds which may reproduce some effects of CR and may also extend maximum life span.

In one embodiment, a study included eight experimental groups as illustrated in Table 1. In one embodiment, the control group was compared to each of the seven treatment groups to determine the specific effects of each treatment on gene expression. It is to be appreciated that the control group can also be compared to each of the seven treatment groups to determine the specific effects of each treatment on nucleic acid levels, protein activity levels, and protein levels. The results from the LT-CR and ST-CR groups were compared to results from each of the treatments of the five test compounds. In one embodiment, these comparisons were used to characterize gene expression profiles common to drug treatments and CR.

To identify differentially expressed genes between any treatment and the control group, each of the four samples in the control group was compared with each of the four samples in the treatment group, resulting in sixteen pairwise comparisons. These data were analyzed statistically using a method based on Wilcoxon's signed rank test. Difference values (PM−MM) between any two groups of arrays were used to generate a one-sided p-value for each set of probes. Default boundaries between significant and not significant p-values were used (See Affymetrix, I. New Statistical Algorithms for Monitoring Gene Expression on GeneChip Probe Arrays, mentioned above, for more details). Genes are considered to have changed expression if the number of increase or decrease calls is 50% or higher in the pairwise comparisons, and an average fold change, derived from all possible pairwise comparisons, is 1.5-fold or greater. Empirically, we found that these criteria identified gene expression changes which were reliably verified by Northern blots, details can further be found in Cao, et. al., Genomic profiling of short-and long-term caloric restriction in the liver of aging mice, Proc. Natl. Acad. Sci. U.S.A. 98, 10630-10635 (2001). The gene expression changes can also be verified by methods such as Western blot, dot blot, primary extension, activity assays, real time PCR, and real time RT-PCR (reverse transcriptase PCR).

Gene names were obtained from the Jackson Laboratory Mouse Genome Infomatics database as of Dec. 1, 2002.

In one embodiment, the effects caused by LT-CR and ST-CR dietary regimens and Metformin, Glipizide, Rosiglitazone, and Soy Isoflavones and combinations thereof are listed in Tables 3-8. These effects are illustrated in terms of gene expression fold changes for various genes. In Table 3, the numbers in the Metformin column represent the average fold change in specific mRNA derived from all 16 possible pairwise comparisons among individual mice from the Metformin and the control (CON) groups (n=4). The numbers in the LT-CR column represent the average fold change in specific mRNA derived from all 16 possible pairwise comparisons among individual mice from the LT-CR and the CON groups (n=4). The numbers in the ST-CR column represent the average fold change in specific mRNA derived from all 16 possible pairwise comparisons among individual mice from the ST-CR and the CON groups (n=4). Where there is no change in gene expression, an “NC” is denoted. Table 4 is similar to Table 3 except it applies to Glipizide. Thus, numbers in the Glipizide column represent the average fold change in specific mRNA derived from all 16 possible pairwise comparisons among individual mice from the Glipizide and the CON groups (n=4). Table 5 is similar to Table 3 except it applies to the Glipizide and Metformin (GM) combination. Thus, numbers in the GM column represent the average fold change in specific mRNA derived from all 16 possible pairwise comparisons among individual mice from the GM combination and the CON groups (n=4). Table 6 is similar to Table 3 except it applies to Rosiglitazone. Thus, numbers in the Rosiglitazone column represent the average fold change in specific mRNA derived from all 16 possible pairwise comparisons among individual mice from the Rosiglitazone and the CON groups (n=4). Table 7 is similar to Table 3 except it applies to Soy Isoflavones. Thus, numbers in the Soy Isoflavone column represent the average fold change in specific mRNA derived from all 16 possible pairwise comparisons among individual mice from the Soy Isoflavone and the CON groups (n=4).

In one embodiment, the fold changes are determined to illustrate the effects on gene expression. If the level of expression of a gene in the treatment groups is equal to or greater than the level of expression in the CON group, the fold change in expression is calculated as a ratio in which the numerator is the level of expression of a gene after one of LT-CR, ST-CR, Metformin, Glipizide, a combination of Metformin and Glipizide, Rosiglitazone, or Soy Isoflavone treatment, and the denominator is the level of expression of the gene in the CON group. For example, the fold change in the expression of a gene in the LT-CR group is the ratio of the expression level of that gene in LT-CR mice to the level of expression of that gene in the CON group; the fold change in the expression of a gene caused by ST-CR is the ratio of the expression of the gene in the ST-CR group to the level of expression of that gene in the CON group; and the fold change in the expression of a gene in the Metformin, Glipizide, a Glipizide Metformin combination, Rosiglitazone, or Soy Isoflavone groups, is the ratio of the expression of a gene in one of the Metformin, Glipizide, a Glipizide Metformin combination, Rosiglitazone, or Soy Isoflavone groups, to the expression level of that gene in the CON group. If the level of expression of a gene in the treatment groups is less than the level of expression in the CON group, the fold change in expression is calculated as the negative inverse of the ratio. Thus, the level of expression of the gene in the CON group is the numerator and the level of expression of that gene in the treatment group is the denominator and a minus sign is used to indicate a decrease in fold change.

In one embodiment, the ability of several glucoregulatory pharmaceuticals (e.g., Metformin, Glipizide, and Rosiglitazone), and other compounds such as Soy Isoflavones to produce CR-specific gene expression profiles in the liver of mice was assessed using the Affymetrix microarrays. The compounds were fed to mice using the mentioned scheme illustrated in FIG. 1.

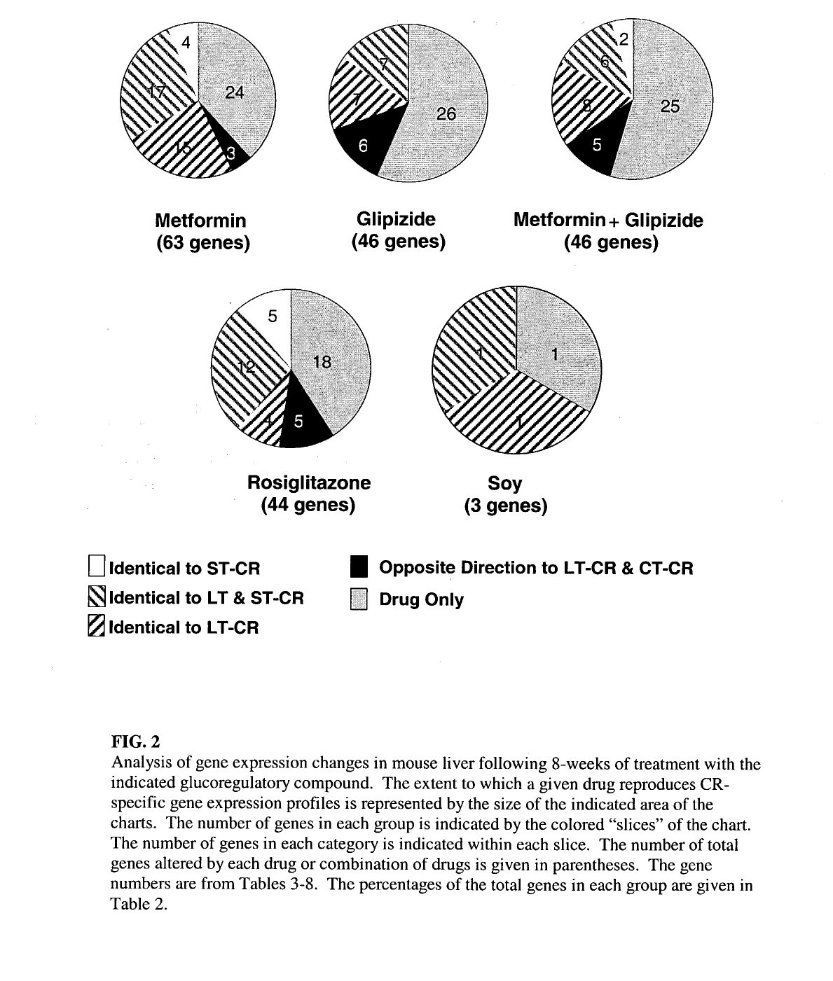

FIG. 2 illustrates that in one embodiment, administering the drugs to mice for eight weeks significantly changed the expression of 63 genes for Metformin, 46 for Glipizide, 46 for a combination of Metformin and Glipizide, 44 for Rosiglitazone, and 3 for Soy Isoflavones. Of the 63 genes with changed expression caused by Metformin: 4 genes with changed expression have identical changes as those caused by ST-CR; 17 genes with changed expression have identical changes as those caused by LT-CR and ST-CR; 15 genes with changed expression have identical changes as those caused by LT-CR; 3 genes with changed expression have the opposite direction of change compared to those caused by LT-CR and ST-CR; and 24 genes with changed expression that are just due to the administration of Metformin alone.

Still with FIG. 2, of the 46 genes with changed expression caused by Glipizide: 0 genes with changed expression have identical changes as those caused by ST-CR; 7 genes with changed expression have identical changes as those caused by LT-CR and ST-CR; 7 genes with changed expression have identical changes as those caused by LT-CR; 6 genes with changed expression have the opposite direction of change compared to those caused by LT-CR and ST-CR; and 26 genes with changed expression that are just due to the administration of Glipizide alone.

Still with FIG. 2, of the 44 genes with changed expression caused by Rosiglitazone: 5 genes with changed expression have identical changes as those caused by ST-CR; 12 genes with changed expression have identical changes as those caused by LT-CR and ST-CR; 4 genes with changed expression have identical changes as those caused by LT-CR; 5 genes with changed expression have the opposite direction of change compared to those caused by LT-CR and ST-CR; and 18 genes with changed expression that are just due to the administration of Rosiglitazone alone.

Still with FIG. 2, of the 46 genes with changed expression caused by the Metformin and Glipizide combination: 2 genes with changed expression have identical changes as those caused by ST-CR; 6 genes with changed expression have identical changes as those caused by LT-CR and ST-CR; 8 genes with changed expression have identical changes as those caused by LT-CR; 5 genes with changed expression have the opposite direction of change compared to those caused by LT-CR and ST-CR; and 25 genes with changed expression that are just due to the administration of Metformin and Glipizide combination alone.

FIG. 2 further illustrates that of the 3 genes that changed expression caused by the administration of Soy Isoflavones, 1 of them is identical to LT-CR, 1 of them is identical to LT-CR and ST-CR, and 1 is due to the administration of Soy Isoflavones alone.

Table 2 summarizes in percentages the extent to which a compound or compound combination reproduces CR-specific gene expression profiles in the results illustrated in FIG. 2. For Metformin, 57% (36 genes) of the induced changes in expression were a subset of the changes induced by either LT- or ST-CR. The other values were 48% (21 genes) for Rosiglitazone, 35% (16 genes) for the combination of Metformin and Glipizide, 30% (14 genes) for Glipizide, and 67% (2 gene) for Soy Isoflavones. These percentages clearly indicate that the glucoregulatory pharmaceuticals substantially reproduce CR-specific gene expression profiles.

Additionally, of the 63 genes altered by Metformin, 51% (32 genes) were changed similarly by LT-CR and 33% (21 genes) by ST-CR (FIG. 2; Table 2). A total of 57% (36 genes) of the Metformin-induced gene expression changes were reproduced with either LT- or ST-CR. Twenty seven percent of the genes whose expression was affected by Metformin were altered by both LT-CR and ST-CR (17 genes). Metformin produced 24 changes in the expression of genes which were not affected by LT- or ST-CR (38% of the changes). Here, we term these effects drug specific changes to distinguish them from the effects in common with CR. Finally, there were 3 genes which Metformin induced to change expression in a direction opposite to that produced by LT-CR (FIG. 2).

Additionally, of the 44 genes altered by Rosiglitazone, 36% (16 genes) were changed similarly by LT-CR and 39% (17 genes) by ST-CR (FIG. 2; Table 2). A total of 48% (21 genes) of the Rosiglitazone-induced gene expression changes were reproduced with either LT- or ST-CR. Twenty seven percent of the genes whose expression was affected by Rosiglitazone were altered by both LT-CR and ST-CR (12 genes). Rosiglitazone produced 18 changes in the expression of genes which were not affected by LT- or ST-CR (41% of the changes). Finally, there were 5 genes which Rosiglitazone induced to change expression in a direction opposite to that produced by LT-CR (FIG. 2).

Additionally, of the 46 genes altered by Glipizide, 30% (14 genes) were changed similarly by LT-CR and 15% (7 genes) by ST-CR (FIG. 2; Table 2). Fifteen percent of the genes whose expression was affected by Glipizide were altered by both LT-CR and ST-CR (7 genes). Glipizide produced 26 changes in the expression of genes which were not affected by LT- or ST-CR (56% of the changes). Finally, there were 6 genes which Glipizide induced to change expression in a direction opposite to that produced by LT-CR (FIG. 2).

Additionally, of the 46 genes altered by the Glipizide-Metformin combination, 30% (14 genes) were changed similarly by LT-CR and 17% (8 genes) by ST-CR (FIG. 2; Table 2). A total of 35% (16 genes) of the Glipizide-Metformin-induced gene expression changes were reproduced with either LT- or ST-CR. Thirteen percent of the genes whose expression was affected by Glipizide-Metformin were altered by both LT-CR and ST-CR (6 genes). Glipizide-Metformin produced 25 changes in the expression of genes which were not affected by LT- or ST-CR (54% of the changes). Finally, there were 5 genes which Glipizide-Metformin induced to change expression in a direction opposite to that produced by LT-CR (FIG. 2).

Additionally, of the 3 genes altered by Soy Isoflavones, 67% (1 gene) was changed similarly by LT-CR and 1 gene which Soy Isoflavones induced to change expression that was not observed in LT-CR or ST-CR (FIG. 2).

As illustrated further in Table 3, the genes that changed expression with Metformin and CR are associated with stress and chaperone proteins, metabolism, signal transduction, and the cytoskeleton. Table 3 indicates the changes in various gene expressions that are caused by Metformin as well as LT-CR and ST-CR. These results indicate that Metformin can be used as a compound that reproduces the effects (or at least some of the effects) of CR including delaying aging and delaying onset of aging related diseases. For example, the expression of glucose 6-phosphatase was induced with Metformin and LT-CR. This is a key enzyme in gluconeogenesis. These results are consistent with other microarray and conventional studies which show that CR increases the enzymatic capacity of the liver for gluconeogenesis and the disposal of the byproducts of extrahepatic protein catabolism for energy production. See for example, Dhahbi, et. al., Caloric restriction alters the feeding response of key metabolic enzyme genes, Mech. Ageing Dev. 122, 35-50, 2001, and Dhahbi, et al., Calories and aging alter gene expression for gluconeogenic, glycolytic, and nitrogen-metabolizing enzymes, Am. J. Physiol. 277, E352-E360, 1999. This CR effect, which is reproduced with Metformin, is consistent with theories of aging, such as the oxidative stress theory, which postulates that the accumulation of damaged proteins contributes to the rate of aging. CR prevents or retards the development of age-related diseases, and extends average and maximum life span in otherwise healthy rodents as well as variety of other species. Metformin, being able to reproduce the key effects to the gene expression mentioned above and as illustrated in Table 3, is expected to be able to, like CR, prevent or retard the development of age-related diseases, and extend average and maximum life span in otherwise healthy rodents as well as variety of other species such as fish, dogs, monkeys, and other mammals including humans.

Furthermore, analysis of genes for which expression is different between the control diet group (e.g., CON group 108) and the CR diet groups (e.g., ST-CR group 110 and LT-CR group 122) can demonstrate that specific genes are preferentially expressed during CR, LT-CR, or ST-CR. The same kind of analysis performed for gene expression that is caused by the test compounds can also be performed. The results which indicate that genes which change expression during treatments with the test compounds, such as Metformin and that are the same genes which change expression during CR, indicate that such compounds can be a CR mimetic compound that reproduces at least some of the effects of CR such as preventing or retarding the development of age-related diseases and extending average and maximum life span in otherwise healthy rodents as well as variety of other species (e.g., humans).

Expression of the molecular chaperone, glucose regulated protein 58 kDa, was decreased with Metformin, and LT- and ST-CR. Studies with microarray analysis have indicated that CR negatively regulates the expression of nearly all endoplasmic reticulum chaperones. Reduced chaperone expression is proapoptotic and anti-neoplastic; elevated chaperone levels tip the balance away from apoptosis and toward cell survival. Thus, there is an inverse correlation between chaperone protein expression and the survival of pre-cancerous cells. Lowering chaperone proteins will tend to reduce cancer incidence. Compounds such as Metformin that reduce chaperone protein expression will tend to reduce the incidence of cancer.

Additionally, chaperone induction has emerged as a new anti-apoptotic mechanism in some cells and tissues. Elevated chaperone levels during tumorigenesis allow cells to survive carcinogenesis and tumor formation. Induced GRP78, GRP94 and GRP170 are essential for the survival, growth and immuno-resistance of transformed cells. Tumorigenesis-associated chaperone induction confers drug resistance to the tumors. Chaperone induction allows precancer cells to survive the DNA damage and mutations which result in transformation, proliferation and onset of carcinogenesis. Metformin reduces chaperone levels in liver and this will tend to reduce the incidence of cancer.

Tables 4-7 illustrate the changes in gene expression caused by Glipizide, a Metformin & Glipizide combination, Rosiglitazone and Soy Isoflavones as well as by LT-CR and ST-CR. These tables include the genes that changed expression with the drug and CR as well as genes that changed expression with the drug only.

Table 8 includes genes whose expression is altered in the opposite direction by LT-CR and the compounds administered to mice.

As can be seen from the results, Rosiglitazone (Table 6) and Glipizide (Table 4) can also be CR mimetics to reproduce the effects (or at least some of the effects) of CR, LT-CR, and/or ST-CR. On the other hand, Soy Isoflavones produce only three changes in gene expression. One change was identical to LT-CR and ST-CR, and one change was identical to LT-CR (Table 7). Soy Isoflavones are putative chemopreventatives. Thus, Soy Isoflavones did not give a strong positive outcome in this assay as did Glipizide, Metformin, a Metformin and Glipizide combination, and Rosiglitazone.

It is to be appreciated that not all effects of CR are desirable. For example, CR suppresses immunity, reduces libido, reduces fertility, and suppresses adrendal and gonadal steroid production. Thus, not all, or indeed, not many of the effects induced by CR need to be reproduced by a test compound such as Metformin in order for the test compound to be recognized as a drug that reproduces beneficial effects of CR.

Various embodiments of the present invention were used to screen several test compounds, e.g., glucoregulatory pharmaceuticals such as Metformin, Glipizide, and Rosiglitazone and Soy Isoflavone extract for their ability to mimic or reproduce the effects of ST-CR and/or LT-CR on gene expression. The glucoregulatory pharmaceuticals, and the combination of two of these pharmaceuticals produced a significant number of changes in hepatic gene expression that are identical to those produced by LT- and/or ST-CR. These findings suggest that these compounds are promising candidate CR-mimetics. Soy Isoflavones did not produce a strongly positive gene-expression signature. These results suggest that microarray profiling is a rapid method of screening drugs for the anti-aging and anti-disease properties. It is expected that Metformin, Glipizide, and Rosiglitazone (and analogous compounds) may be administered at effective dosages, to mammals including humans, to reproduce at least some of the effects of CR. Furthermore, Metformin, Glipizide, and Rosiglitazone(and analogous compounds) may be administered to mammals, including humans and mice, to increase the maximum life span of an otherwise healthy mammal. The analogous compounds include derivatives (e.g., salt derivatives) and other chemically similar structures. The effective dosages for Metformin may be approximately between 0.2 mg and 2.0 gm of Metformin per kg body weight per day. The effective dosages for Glipizide may be approximately between 1.05×10⁻3 mg and 105 mg of Glipizide per kg body weight per day. The effective dosages for Rosiglitazone may be approximately between 8.0×10⁻4 mg and 8 mg of Rosiglitazone per kg body weight per day. The effective dosages for the combination of Metformin and Glipizide may be approximately between 0.1 mg and 1.0 gm per kg body weight per day of Metformin plus approximately between 0 mg and 52.5 mg of Glipizide per kg body weight per day.

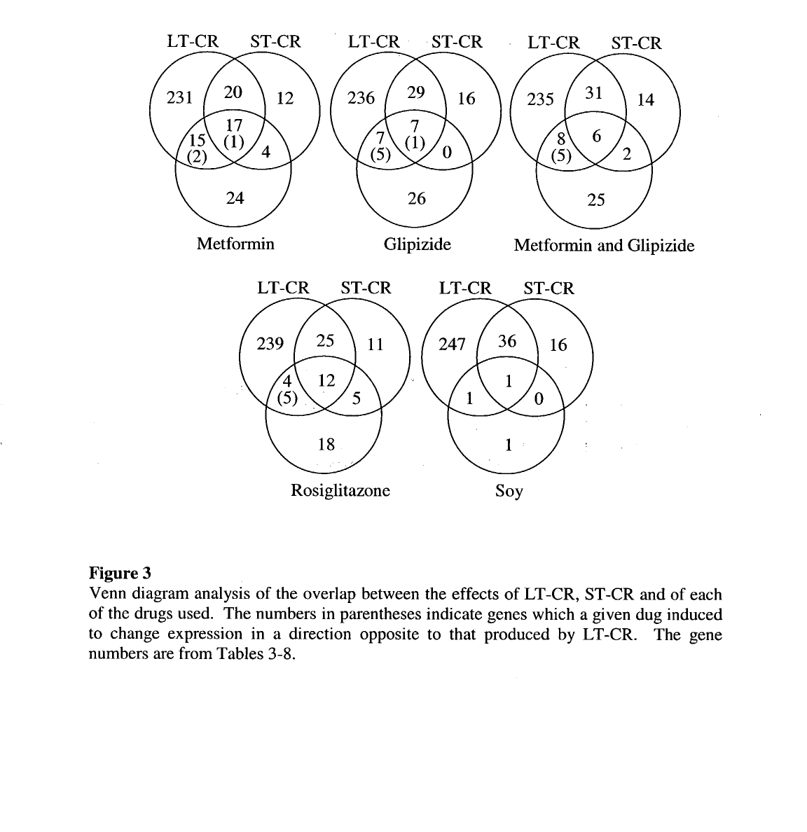

In one embodiment, the gene expression profiles induced by the different compounds or drugs are compared to the gene expression profiles induced by LT- and ST-CR to identify the common changes in gene expression and to determine the extent to which the drugs reproduce CR specific effects. The extent to which each of the tested compound (e.g., Metformin, Glipizide, Rosiglitazone, and Soy Isoflavones) reproduced the effects of CR on gene expression was determined. FIG. 3 illustrates a Venn diagram analysis of the overlap between the effects of LT-CR, ST-CR, and of each of the compounds or drugs administered to the test groups as shown in FIG. 1. The numbers in parentheses indicate genes which a given drug induced to change expression in a direction opposite to that produced by LT-CR. The gene numbers are from Tables 3-8. As illustrated in Table 9 and FIG. 3, Metformin reproduced 11.3% (32 out of 283 genes) of the effects of LT-CR on gene expression. Metformin reproduced 39.6% (21 out of 53 genes) of the effects of ST-CR on gene expression. Glipizide reproduced 5.0% (14 out of 279 genes) of the effects of LT-CR on gene expression. Glipizide reproduced 13.5% (7 out of 52 genes) of the effects of ST-CR on gene expression. The combination of Metformin and Glipizide reproduced 5.0% (14 out of 280 genes) of the effects of LT-CR on gene expression. The combination of Metformin and Glipizide reproduced 15.1% (8 out of 51 genes) of the effects of ST-CR on gene expression. Rosiglitazone reproduced 5.7% (16 out of 280 genes) of the effects of LT-CR on gene expression. Rosiglitazone reproduced 32.1% (17 out of 48 genes) of the effects of ST-CR on gene expression. Soy Isoflavones reproduced 0.7% (2 out of 285 genes) of the effects of LT-CR on gene expression. Soy Isoflavones reproduced 0% (1 out of 53 genes) of the effects of ST-CR on gene expression. These percentages clearly indicate that Metformin, Glipizide, and Rosiglitazone share several common effects on hepatic gene expression with CR. As can be seen, Metformin is more effective in reproducing some of the effects of CR than Glipizide, Rosiglitazone, and a Glipizide-Metformin combination. Soy Isoflavones are not effective in reproducing effects of CR as were the other tested compounds.

The various methods described herein may be used to search for (e.g., screen) drug candidates (e.g., an intervention), which can reproduce at least some of the effects of CR (e.g., either ST-CR or LT-CR) in mammals, including humans. Further, these methods may be used to search for (e.g., screen) drug candidates (e.g., an intervention), which can extend the maximum life span of an organism, including a human.

It can be expected that agents, identified in the embodiments described above, will extend lifespan, delay aging related diseases, and increase the age of onset and reduce the incidence of age-related diseases. Agents which reproduce the LT-CR or ST-CR signature (e.g., a similar pattern of gene expression changes) in microarray assays or other assays are likely to act as authentic CR mimetics and to extend maximum lifespan and improve health generally by delaying the onset and reducing the incidence of age related diseases.

While particular embodiments of the present invention have been shown and described, it will be obvious to those skilled in the art that changes and modifications can be made without departing from this invention in its broader aspects and, therefore, the appended claims are to encompass within their scope all such changes and modifications as fall within the scope of this invention.

TABLE 1
Experimental Groups.

| Group | Drug or diet |
|---|---|
| 1 | Metformin (2100) |
| 2 | Glipizide (1050) |
| 3 | Metformin (1050) & Glipizide (525) |
| 4 | Rosiglitazone (80) |
| 5 | Soy (.25%) |
| 6 | Long-term calorie restriction |
| 7 | Short-term calorie restriction (8 weeks) |
| 8 | Control |

Notes:
Numbers in parentheses indicate the amount of each compound in mg/kilogram of the control diet, unless otherwise indicated.

TABLE 2
Percentage of drug-specific effects and overlap between the
effects of CR and those of each of the drugs used.

| | | Glip- | Metformin & | | |
|---|---|---|---|---|---|
| | Metformin | izide | Glipizide | Rosiglitazone | Soy |
| LT- or ST-CR | 57% | 30% | 35% | 48% | 67% |
| LT-CR | 51% | 30% | 30% | 36% | 67% |
| ST-CR | 33% | 15% | 17% | 39% | 33% |
| LT- and ST-CR | 27% | 15% | 13% | 27% | 33% |
| Drug-specific | 38% | 57% | 54% | 41% | 33% |

TABLE 3
Effects of Metformin and CR on hepatic gene expression.

| | | | | |
|---|---|---|---|---|
| Gene/Protein | GenBank | Metformin¹ | LT-CR^{2,4} | ST-CR^{3,4} |

Changes in gene expression induced by Metformin and reproduced with either LT- or ST-CR

| | | | | |
|---|---|---|---|---|
| Stress and chaperone proteins | | | | |
| Cytochrome P450, 2b13, phenobarbitol | M60358 | 3.4 | 2.7 | 1.8 |
| inducible, type c | | | | |
| Cytochrome P450, 4a12 | Y10221 | −3.1 | −3.2 | −2.8 |
| ATP-binding cassette, sub-family G | AF103875 | −1.5 | −1.6 | NC |
| (WHITE), member 2 | | | | |
| Metallothionein 2 | K02236 | −1.9 | −4.4 | NC |
| Glucose regulated protein, 58 kDa | M73329 | −1.5 | −1.6 | −1.5 |
| Heat shock 70 kD protein 5 (glucose- | AJ002387 | −1.5 | −1.8 | −1.5 |
| regulated protein, 78 kD) | | | | |
| Metabolism | | | | |
| Farnesyl pyrophosphate synthase | AI846851 | 3.1 | 1.5 | 1.7 |
| Farnesyl pyrophosphate synthase (Second | AW045533 | 3.7 | 1.5 | 1.5 |
| time) | | | | |
| Fatty acid synthase | X13135 | 2.4 | NC | 1.6 |
| ATP-binding cassette, sub-family A (ABC1), | AI845514 | −1.5 | −1.5 | NC |
| member 1 | | | | |
| Glucose-6-phosphatase, catalytic | U00445 | 1.6 | 2.8 | NC |
| Aquaporin 1 | L02914 | 1.6 | 1.5 | NC |
| Arylsulfatase A | X73230 | −1.7 | −2.4 | −2.2 |
| Arylsulfatase A (second time) | AF109906 | 1.8 | 4.6 | NC |
| Cytoskeleton | | | | |
| keratin complex 1, acidic, gene 18 | M22832 | −1.7 | −1.7 | −1.5 |
| Keratin complex 2, basic, gene 8 | X15662 | −1.5 | −2.2 | −1.7 |
| Actin, gamma, cytoplasmic | M21495 | −1.5 | −3.2 | −2.1 |
| Actin, beta, cytoplasmic | M12481 | −1.6 | −1.5 | NC |
| Vinculin | AI462105 | −1.5 | −1.6 | NC |
| Signal Transduction | | | | |
| Ectonucleotide | AW122933 | −1.5 | −2.9 | −1.5 |
| pyrophosphatase/phosphodiesterase 2 | | | | |
| Dual specificity phosphatase 1 | X61940 | 1.5 | 1.7 | NC |
| Suppressor of cytokine signaling 2 | U88327 | 1.6 | 1.9 | 1.7 |
| Interferon gamma induced GTPase | U53219 | −1.7 | −3.1 | −1.7 |
| Interferon-g induced GTPase | AJ007972 | −1.5 | −2.7 | −1.7 |
| Interferon-inducible GTPase | AA914345 | −1.7 | −2.9 | −1.5 |
| Interferon-inducible GTPase (second copy) | AJ007971 | −1.6 | −2.7 | −1.6 |
| Pre B-cell leukemia transcription factor 1 | AW124932 | 1.8 | NC | 1.5 |
| Regulator of G-protein signaling 16 | U94828 | 2.0 | NC | 1.6 |
| Activating transcription factor 3 | U19118 | −1.9 | −1.8 | −1.5 |
| Cholinergic receptor, nicotinic, beta | AI842969 | −1.5 | −1.7 | NC |
| polypeptide 3 | | | | |
| Miscellaneous | | | | |
| Complement component 9 | X05475 | −1.5 | −2.1 | NC |
| Hermansky-Pudlak syndrome 1 homolog | AI551087 | −1.6 | −1.5 | NC |
| (human) | | | | |
| Major urinary protein 1 | AI255271 | −1.6 | NC | −1.5 |
| EST | C79248 | −1.6 | −1.7 | NC |
| EST | AI787317 | −1.6 | −1.7 | NC |
| EST | AA690218 | 1.5 | 2.6 | NC |

Metformin-specific changes in gene expression

| | | | | |
|---|---|---|---|---|
| Energy metabolism | | | | |
| Pyruvate kinase liver and red blood cell | D63764 | 1.8 | NC | NC |
| Glucokinase | L41631 | 1.6 | NC | NC |
| Diaphorase 1 (NADH)(cytochrome b-5 | AW122731 | 1.5 | NC | NC |
| reductase) | | | | |
| Guanidinoacetate methyltransferase | AF010499 | 1.5 | NC | NC |
| NAD(P) dependent steroid dehydrogenase- | AW106745 | 1.9 | NC | NC |
| like | | | | |
| Phospholipid transfer protein | U28960 | 1.8 | NC | NC |
| Thyroid hormone responsive SPOT14 | X95279 | 2.4 | NC | NC |
| homolog (Rattus) | | | | |
| Trans-golgi network protein 2 | AA614914 | −1.5 | NC | NC |
| Glutathione S-transferase, alpha 2 (Yc2) | J03958 | −1.5 | NC | NC |
| NAD(P) dependent steroid dehydrogenase- | AL021127 | 2.0 | NC | NC |
| like | | | | |
| Transketolase | U05809 | 1.5 | NC | NC |
| Signal transduction | | | | |
| Programmed cell death 4 | D86344 | −1.6 | NC | NC |
| Protein phosphatase 1, catalytic subunit, beta | M27073 | −1.5 | NC | NC |
| isoform | | | | |
| Diazepam binding inhibitor | X61431 | 1.7 | NC | NC |
| Enolase 1, alpha non-neuron | AI841389 | 1.5 | NC | NC |
| Miscellaneous | | | | |
| Ia-associated invariant chain | X00496 | 1.5 | NC | NC |
| Murinoglobulin 1 | M65736 | −1.5 | NC | NC |
| Zinc finger protein 265 | AI835041 | −1.6 | NC | NC |
| EST | AI853364 | 1.7 | NC | NC |
| EST | AI852741 | −1.5 | NC | NC |
| EST | AV291989 | −1.5 | NC | NC |
| EST | AA733664 | −1.5 | NC | NC |
| EST | AW212131 | −1.5 | NC | NC |
| EST | AW124226 | −1.6 | NC | NC |

¹The numbers in this column represent the average fold change in specific mRNA derived from all 16 possible pairwise comparisons among individual mice from Metformin and CON groups (n = 4).
²The numbers in this column represent the average fold change in specific mRNA derived from all 16 possible pairwise comparisons among individual mice from LT-CR and CON groups (n = 4).
³The numbers in this column represent the average fold change in specific mRNA derived from all 16 possible pairwise comparisons among individual mice from ST-CR and CON groups (n = 4).
⁴“NC” indicates no change in gene expression.

TABLE 4
Effects of Glipizide and CR on hepatic gene expression.

| | | | | |
|---|---|---|---|---|
| Gene/Protein | GenBank | Glipizide¹ | LT-CR^{2,4} | ST-CR^{3,4} |

Changes in gene expression induced by Glipizide and reproduced with either LT- or ST-CR

| | | | | |
|---|---|---|---|---|
| Stress and chaperone proteins | | | | |
| Heat shock protein, 105 kDa | L40406 | 1.7 | 2.3 | NC |
| Cytochrome P450, 4a12 | Y10221 | −1.9 | 3.2 | −2.8 |
| ATP-binding cassette, sub-family G | AF103875 | −1.5 | −1.6 | NC |
| (WHITE), member 2 | | | | |
| Metabolism | | | | |
| Vanin 1 | AJ132098 | −1.5 | −1.6 | −1.5 |
| Ectonucleotide | AW122933 | −1.6 | −2.9 | −1.5 |
| pyrophosphatase/phosphodiesterase 2 | | | | |
| Retinoic acid early transcript gamma | D64162 | −1.5 | −3.1 | NC |
| Hydroxysteroid dehydrogenase-6, delta<5>- | AF031170 | −1.6 | −1.5 | NC |
| 3-beta | | | | |
| Signal Transduction | | | | |
| Suppressor of cytokine signaling 2 | U88327 | 2.0 | 1.9 | 1.7 |
| Complement component 2 (within H-2S) | AF109906 | 1.8 | 4.6 | NC |
| Activating transcription factor 3 | U19118 | −2.0 | −1.8 | −1.5 |
| Cytoskeleton | | | | |
| Actin, gamma, cytoplasmic | M21495 | −1.7 | −3.2 | −2.1 |
| Miscellaneous | | | | |
| Lectin, galactose binding, soluble 1 | X15986 | −1.7 | −2.6 | −1.8 |
| EST | AA959954 | −1.5 | −2.2 | NC |
| EST | AI266885 | −1.7 | −1.6 | NC |

Glipizide-specific changes in gene expression

| | | | | |
|---|---|---|---|---|
| Stress and chaperone proteins | | | | |
| Cytochrome P450, 1a2, aromatic compound | X04283 | 1.6 | NC | NC |
| inducible | | | | |
| Cytochrome P450, 4a10 | AB018421 | −1.7 | NC | NC |
| Cytochrome P450, 4a14 | Y11638 | −1.5 | NC | NC |
| DnaJ (Hsp40) homolog, subfamily C, | U28423 | 1.6 | NC | NC |
| member 3 | | | | |
| Metabolism | | | | |
| Stearoyl-Coenzyme A desaturase 1 | M21285 | −1.8 | NC | NC |
| Hydroxysteroid dehydrogenase-3, delta<5>- | M77015 | −1.5 | NC | NC |
| 3-beta | | | | |
| Thyroid hormone responsive SPOT14 | X95279 | −1.7 | NC | NC |
| homolog (Rattus) | | | | |
| Glutathione S-transferase, alpha 2 (Yc2) | J03958 | −1.6 | NC | NC |
| Cathepsin C | U74683 | 1.5 | NC | NC |
| DNA cross-link repair 1A, PSO2 homolog | AI225445 | −1.5 | NC | NC |
| (S. cereviciae) | | | | |
| Signal transduction | | | | |
| Activating transcription factor 5 | AB012276 | 1.5 | NC | NC |
| Hepcidin antimicrobial peptide | AI255961 | 1.5 | NC | NC |
| Angiogenin | U22516 | 1.5 | NC | NC |
| Butyrylcholinesterase | M99492 | −1.5 | NC | NC |
| Wee 1 homolog (S. pombe) | D30743 | −1.5 | NC | NC |
| Miscellaneous | | | | |
| Staphylococcal nuclease domain containing 1 | AB021491 | 1.5 | NC | NC |
| Pre-B-cell colony-enhancing factor | AI852144 | −1.5 | NC | NC |
| Complement component 1, q subcomponent, | X58861 | 1.5 | NC | NC |
| alpha polypeptide | | | | |
| EST | AA612450 | −1.5 | NC | NC |
| EST | AA959954 | −1.5 | NC | NC |
| EST | AI850090 | −1.5 | NC | NC |
| EST | AI852184 | 1.6 | NC | NC |
| EST | AW047688 | −1.5 | NC | NC |
| EST | AW060549 | −1.6 | NC | NC |
| EST | AW122942 | 1.5 | NC | NC |
| EST | AW212131 | −1.5 | NC | NC |

¹The numbers in this column represent the average fold change in specific mRNA derived from all 16 possible pairwise comparisons among individual mice from Glipizide and CON groups (n = 4).
²The numbers in this column represent the average fold change in specific mRNA derived from all 16 possible pairwise comparisons among individual mice from LT-CR and CON groups (n = 4).
³The numbers in this column represent the average fold change in specific mRNA derived from all 16 possible pairwise comparisons among individual mice from ST-CR and CON groups (n = 4).
⁴“NC” indicates no change in gene expression.

TABLE 5
Effects of Glipizide & Metformin (GM) and CR on hepatic gene expression.

| | | | | |
|---|---|---|---|---|
| Gene/Protein | GenBank | GM¹ | LT-CR^{2,4} | ST-CR^{3,4} |

Changes in gene expression induced by GM and reproduced with either LT- or ST-CR

| | | | | |
|---|---|---|---|---|
| Stress and chaperone proteins | | | | |
| Heat shock protein, 105 kDa | L40406 | 1.7 | 2.3 | NC |
| DnaJ (Hsp40) homolog, subfamily B, | AB028272 | 1.5 | 1.6 | NC |
| member 1 | | | | |
| Cytochrome P450, 4a12 | Y10221 | −1.5 | 3.2 | −2.8 |
| Metabolism | | | | |
| Farnesyl pyrophosphate synthase | AI846851 | 1.5 | 1.5 | 1.7 |
| Farnesyl pyrophosphate synthase (Second | AW045533 | 1.8 | 1.5 | 1.5 |
| time) | | | | |
| Retinoic acid early transcript gamma | D64162 | −1.6 | −3.1 | NC |
| Sialyltransferase 9 (CMP- | | | | |
| NeuAc:lactosylceramide alpha-2,3- | Y15003 | 1.6 | 2.5 | NC |
| sialyltransferase; GM3 synthase) | | | | |
| Signal Transduction | | | | |
| Suppressor of cytokine signaling 2 | U88327 | 2.7 | 1.9 | 1.7 |
| Complement component 2 (within H-2S) | AF109906 | 2.0 | 4.6 | NC |
| Regulator of G-protein signaling 16 | AV349152 | 1.5 | NC | 1.6 |
| Regulator of G-protein signaling 16 | U94828 | 1.7 | NC | 1.6 |
| Angiopoietin-like 4 | AA797604 | 1.6 | 1.8 | NC |
| Insulin-like growth factor binding protein 1 | X81579 | 1.5 | 2.4 | NC |
| Cytoskeleton | | | | |
| Actin, gamma, cytoplasmic | M21495 | −1.7 | −3.2 | −2.1 |
| Miscellaneous | | | | |
| Lectin, galactose binding, soluble 1 | X15986 | −1.6 | −2.6 | −1.8 |
| EST | AI266885 | −1.5 | −1.6 | NC |

GM-specific changes in gene expression

| | | | | |
|---|---|---|---|---|
| Stress and chaperone proteins | | | | |
| Cytochrome P450, 2b10, phenobarbitol | M21856 | −1.6 | NC | NC |
| inducible, type b | | | | |
| DnaJ (Hsp40) homolog, subfamily C, | U28423 | 1.6 | NC | NC |
| member 3 | | | | |
| Serum amyloid P-component | M23552 | 1.5 | NC | NC |
| Metabolism | | | | |
| 3′-phosphoadenosine 5′-phosphosulfate | AF052453 | −1.5 | NC | NC |
| synthase 2 | | | | |
| Glutathione 5-transferase, alpha 2 (Yc2) | J03958 | −2.0 | NC | NC |
| Phospholipid transfer protein | U28960 | −1.5 | NC | NC |
| Stearoyl-Coenzyme A desaturase 1 | M21285 | −1.9 | NC | NC |
| Thyroid hormone responsive SPOT14 | X95279 | −1.6 | NC | NC |
| homolog (Rattus) | | | | |
| Cytochrome c oxidase, subunit VIc | AV071102 | −1.6 | NC | NC |
| DNA cross-link repair 1A, PSO2 homolog | AI225445 | −1.5 | NC | NC |
| (S. cereviciae) | | | | |
| Signal transduction | | | | |
| Angiogenin | U22516 | 1.6 | NC | NC |
| Bcl2-associated athanogene 3 | AI643420 | 1.6 | NC | NC |
| Prolactin receptor | D10214 | 1.5 | NC | NC |
| Transducin-like enhancer of split 1, homolog | U61362 | 1.5 | NC | NC |
| of Drosophila E(spl) | | | | |
| Deoxyribonuclease II alpha | AW120896 | 1.5 | NC | NC |
| cAMP-regulated guanine nucleotide | AF115480 | 1.5 | NC | NC |
| exchange factor II | | | | |
| Wee 1 homolog (S. pombe) | D30743 | −1.6 | NC | NC |
| Cytoskeleton | | | | |
| Reelin | U24703 | −1.6 | NC | NC |
| Miscellaneous | | | | |
| Butyrylcholinesterase | M99492 | −1.5 | NC | NC |
| Lysophospholipase 1 | AA840463 | −1.5 | NC | NC |
| Leucine-rich alpha-2-glycoprotein | AW23089 | 1.5 | NC | NC |
| Dynein, cytoplasmic, light chain 1 | AF020185 | 1.5 | NC | NC |
| EST | C79676 | −1.5 | NC | NC |
| EST | AI842968 | −1.6 | NC | NC |
| EST | AW124226 | −1.7 | NC | NC |

¹The numbers in this column represent the average fold change in specific mRNA derived from all 16 possible pairwise comparisons among individual mice from GM and CON groups (n = 4).
²The numbers in this column represent the average fold change in specific mRNA derived from all 16 possible pairwise comparisons among individual mice from LT-CR and CON groups (n = 4).
³The numbers in this column represent the average fold change in specific mRNA derived from all 16 possible pairwise comparisons among individual mice from ST-CR and CON groups (n = 4).
⁴“NC” indicates no change in gene expression.

TABLE 6
Effects of Rosiglitazone and CR on hepatic gene expression.

| | | | | |
|---|---|---|---|---|
| Gene/Protein | GenBank | Rosiglitazone¹ | LT-CR^{2,4} | ST-CR^{3,4} |

Changes in gene expression induced by Rosiglitazone and reproduced with either LT- or ST-CR

| | | | | |
|---|---|---|---|---|
| Stress and chaperone proteins | | | | |
| Cytochrome P450, 2f2 | M77497 | −1.6 | −1.5 | −1.5 |
| Cytochrome P450, 2b13, phenobarbitol | M60358 | 1.9 | 2.7 | 1.8 |
| inducible, type c | | | | |
| Cytochrome P450, 4a12 | Y10221 | −2.9 | 3.2 | −2.8 |
| Cytochrome P450, 7a1 | L23754 | −1.7 | −1.7 | NC |
| Metabolism | | | | |
| Ectonucleotide | AW122933 | −1.8 | −2.9 | −1.5 |
| Pyrophosphatase/phosphodiesterase 2 | | | | |
| Apolipoprotein A-IV | M64248 | −3.4 | NC | −1.8 |
| Signal Transduction | | | | |
| Activating transcription factor 3 | U19118 | −1.5 | −1.8 | −1.5 |
| Cytokine inducible SH2-containing protein 2 | U88327 | 1.7 | 1.9 | 1.7 |
| Inhibitor of DNA binding 3 | M60523 | −1.7 | NC | −1.5 |
| Regulator of G-protein signaling 16 | AV349152 | 1.6 | NC | 1.6 |
| Regulator of G-protein signaling 16 | U94828 | 1.8 | NC | 1.6 |
| Cytoskeleton | | | | |
| Actin, gamma, cytoplasmic | M21495 | −1.8 | −3.2 | −2.1 |
| Keratin complex 1, acidic, gene 18 | M22832 | −1.6 | −1.7 | −1.5 |
| Keratin complex 2, basic, gene 8 | X15662 | −1.7 | −2.2 | −1.7 |
| Tubulin, beta 2 | M28739 | −1.5 | NC | −1.5 |
| Miscellaneous | | | | |
| Lectin, galactose binding, soluble 1 | X15986 | −1.8 | −2.6 | −1.8 |
| Arylsulfatase A | X73230 | −1.6 | −2.4 | −2.2 |
| Macrophage expressed gene 1 | L20315 | −1.6 | −2.4 | −1.9 |
| Quiescin Q6 | AW04575 | 1.6 | 1.6 | NC |
| EST | AI530403 | 1.5 | 1.7 | NC |
| EST | AI266885 | −2.0 | −1.6 | NC |

Rosiglitazone-specific changes in gene expression

| | | | | |
|---|---|---|---|---|
| Stress and chaperone proteins | | | | |
| Cytochrome P450, 8b1, sterol 12 alpha- | AF090317 | −1.5 | NC | NC |
| hydrolase | | | | |
| Metabolism | | | | |
| Glutathione S-transferase, alpha 2 (Yc2) | J03958 | −1.7 | NC | NC |
| Flavin containing monooxygenase 5 | U90535 | −1.5 | NC | NC |
| Thyroid hormone responsive SPOT14 | X95279 | −1.5 | NC | NC |
| homolog (Rattus) | | | | |
| Amine N-sulfotransferase | AF026073 | −1.5 | NC | NC |
| DNA cross-link repair 1A, PSO2 homolog | AI225445 | −1.6 | NC | NC |
| (S. cereviciae) | | | | |
| Cathepsin C | U74683 | 1.7 | NC | NC |
| Cathepsin C (second time) | AI842667 | 1.7 | NC | NC |
| Signal transduction | | | | |
| G0/G1 switch gene 2 | X95280 | 1.5 | NC | NC |
| Cytoskeleton | | | | |
| Inter-alpha trypsin inhibitor, heavy chain 3 | X70393 | 1.5 | NC | NC |
| Miscellaneous | | | | |
| Orphan nuclear receptor; Rev-ErbA-alpha | AI834950 | 1.5 | NC | NC |
| protein | | | | |
| RAD51-like 1 (S. cereviciae) | U92068 | 1.5 | NC | NC |
| Pre-B-cell colony-enhancing factor | AI852144 | −1.5 | NC | NC |
| Hemoglobin, beta adult minor chain | V00722 | 1.5 | NC | NC |
| Quiescin Q6 | AW123556 | 1.7 | NC | NC |
| EST | AA619207 | −1.7 | NC | NC |
| EST | AA959954 | −1.5 | NC | NC |
| EST | AW060549 | −1.7 | NC | NC |

¹The numbers in this column represent the average fold change in specific mRNA derived from all 16 possible pairwise comparisons among individual mice from Rosiglitazone and CON groups (n = 4).
²The numbers in this column represent the average fold change in specific mRNA derived from all 16 possible pairwise comparisons among individual mice from LT-CR and CON groups (n = 4).
³The numbers in this column represent the average fold change in specific mRNA derived from all 16 possible pairwise comparisons among individual mice from ST-CR and CON groups (n = 4).
⁴“NC” indicates no change in gene expression.

TABLE 7
Effects of Soy Isoflavone and CR on hepatic gene expression.

| | | | | |
|---|---|---|---|---|
| | | Soy | | |
| Gene/Protein | GenBank | Isoflavone¹ | LT-CR^{2,4} | ST-CR^{3,4} |

Changes in gene expression induced by Soy and
reproduced with either LT- or ST-CR

| | | | | |
|---|---|---|---|---|
| Immunoglobulin kappa | | | | |
| chain variable 28 | | | | |
| (V28) | M18237 | −1.8 | −2.0 | 1.5 |
| EST | M80423 | −2.1 | −2.0 | NC |

Soy-specific changes in gene expression

| | | | | |
|---|---|---|---|---|
| EST | V00817 | −1.5 | NC | NC |

¹The numbers in this column represent the average fold change in specific mRNA derived from all 16 possible pairwise comparisons among individual mice from Soy and CON groups (n = 4).
²The numbers in this column represent the average fold change in specific mRNA derived from all 16 possible pairwise comparisons among individual mice from LT-CR and CON groups (n = 4).
³The numbers in this column represent the average fold change in specific mRNA derived from all 16 possible pairwise comparisons among individual mice from ST-CR and CON groups (n = 4).
⁴“NC” indicates no change in gene expression.

TABLE 8
Genes whose expression is altered in the opposite
direction by LT-CR and the drugs used.

| | | | |
|---|---|---|---|
| Gene/Protein | GenBank | LT- CR¹ | DRUG² |

Metformin

| | | | |
|---|---|---|---|
| Cytochrome P450, 7a1 | L23754 | −1.7 | 1.8 |
| Sterol-C4-methyl oxidase-like | AI848668 | −2.4 | 2.4 |
| EST | AI844396 | −1.6 | 1.9 |

Glipizide

| | | | |
|---|---|---|---|
| Splicing factor 3b, subunit 1, 155 | AI844532 | −1.5 | 1.5 |
| kDa | | | |
| EST | AJ011864 | 1.6 | −1.7 |
| Arginine-rich, mutated in early stage | AW122364 | −1.7 | 1.5 |
| tumors | | | |
| Neuropilin | D50086 | −1.6 | 1.5 |
| Calcium binding protein, intestinal | Y00884 | −1.5 | 1.9 |
| Phosphatase and tensin homolog | U92437 | −1.5 | 1.6 |

Glipizide & Metformin

| | | | |
|---|---|---|---|
| Calcium binding protein, intestinal | Y00884 | −1.5 | 1.7 |
| Metallothionein 1 | V00835 | −4.1 | 1.6 |
| Splicing factor 3b, subunit 1, 155 | AI844532 | −1.5 | 1.5 |
| kDa | | | |
| Carbon catabolite repression 4 | AW047630 | −1.5 | 1.5 |
| homolog (S. cereviciae) | | | |
| Serum amyloid A 1 | M13521 | −1.5 | 2.1 |

Rosiglitazone

| | | | |
|---|---|---|---|
| metallothionein 2 | K02236 | −4.4 | 1.6 |
| insulin-like growth factor binding | X81579 | 2.4 | −1.6 |
| protein 1 | | | |
| metallothionein 1 | V00835 | −4.1 | 1.7 |
| calcium binding protein, intestinal | Y00884 | −1.5 | 1.6 |
| Phosphatase and tensin homolog | U92437 | −1.5 | 1.5 |

¹The numbers in this column represent the average fold change in specific mRNA derived from all 16 possible pairwise comparisons among individual mice from LT-CR and CON groups (n = 4).
²The numbers in this column represent the average fold change in specific mRNA derived from all 16 possible pairwise comparisons among individual mice from a drug and CON groups (n = 4).

TABLE 9
Percentage of CR effects reproduced
by the different drug treatments.

| | LT-CR | ST-CR |
|---|---|---|
| Metformin | 11.3% | 39.6% |
| Glipizide | 5.0% | 13.5% |
| Metformin & | 5.0% | 15.1% |
| Glipizide | | |
| Rosiglitazone | 5.7% | 32.1% |
| Soy | 0.7% | 0% |

We claim:

1. A method of reproducing at least one effect in mammals that have been subjected to long-term caloric restriction (LT-CR) comprising:
administering a LT-CR diet program to a first group of mammals for a first duration of time;
administering at least one compound to a second group of mammals for a second duration of time wherein said second duration of time is substantially shorter than said first duration of time, said first group of mammals and said second group of mammals being similar;
obtaining control data from an administering of a control diet program;
determining effects of said LT-CR diet program and said at least one compound by comparing data obtained for said first group of mammals and said second group of mammals to said control data; and
comparing effects between said LT-CR diet program and said at least one compound to determine whether said at least one compound reproduces at least one effect caused by said LT-CR.

2. A method of claim 1 wherein said first duration of time is about 80 weeks.

3. A method of claim 1 wherein said second duration of time is about 1-8 weeks.

4. A method of claim 1 wherein said compound being a glucoregulatory agent.

5. A method of claim 1 wherein said compound includes at least one of Metformin, Glipizide, Rosiglitazone, Soy Isoflavones, and a combination thereof.

6. A method of claim 1 wherein said comparing effects including comparing at least one of changes in gene expression, changes in levels of nucleic acids, changes in proteins, and changes in protein activity levels.

7. A method of claim 1 wherein said mammals include mice.

8. A method of claim 1 wherein said effects include at least one of extending life of said mammals that are otherwise healthy and delaying onset of age related diseases.

9. A method of claim 1 wherein said effects include at least one of extending life of mice that are otherwise healthy and delaying onset of age related diseases in mice.

10. A method of claim 1 wherein said comparing effects between said LT-CR diet program and said at least one compound including comparing changes in gene expression wherein said changes in gene expression include the genes listed in Tables 3, 4, 5, 6, 7, and 8.

11. A method of extending longevity (or increasing maximum life span) for a mammal that is otherwise healthy comprising:
administering an effective dosage of at least one of Metformin, Glipizide, Rosiglitazone, and Soy Isoflavones to said mammal for an effective amount of time.

12. A method of claim 11 wherein said administering includes adding said effective dosage of said at least one of Metformin, Glipizide, Rosiglitazone, and Soy Isoflavones into diet given to said mammal.

13. A method of claim 11 wherein said mammal includes (laboratory) mice.

14. A method of claim 11 wherein said effective amount of time is about 1 day to about 8 weeks.

15. A method of claim 11 wherein said administering being designed to delay age related diseases.

16. A method of claim 11 wherein said effective dosage being between about 0.2 mg to about 2 g per kg body weight per day for Metformin, about 1.05×10⁻3 mg to about 105 mg per kg body weight per day for Glipizide, about 8×10⁻4 mg per to about 8 mg per kg body weight per day for Rosiglitazone, and about 0.025% to about 2.5% (by weight) for Soy Isoflavones.

17. A method of identifying a compound that reproduces effects of a CR comprising:
administering an effective dosage of a test compound to a first mammal for a duration of time;
administering a CR diet program to a second mammal, said first mammal and said second mammal being similar;
analyzing changes in gene expression levels, levels of nucleic acids, protein, or protein activity levels, in each of said first mammal and said second mammal; and
identifying said test compound as one that reproduces changes induced by said CR when said test compound produces analyzed changes in said first mammal wherein at least about 1% or one or more gene changes of said analyzed changes are a subset of said changes induced by said CR.

18. A method of claim 17 further comprises, obtaining control data from an administering of a control diet program; and said identifying further comprises comparing each of said changes in gene expression levels, levels of nucleic acids, protein, or protein activity levels in each of said first mammal and said second mammal to said control data, and comparing said changes in gene expression levels, levels of nucleic acids, protein, or protein activity levels of said first mammal and said second mammal to each other.

19. A method of claim 17 wherein said CR includes LT-CR and ST-CR.

20. A method of claim 17 wherein said CR is LT-CR wherein said second mammal is subjected to LT-CR for about several months to about end of life wherein said test compound is administered to said first mammal for about 1 day to about 8 weeks.

21. A method of claim 17 wherein said CR is LT-CR wherein said second mammal is subjected to LT-CR for longer than when said test compound is administered to said first mammal.

22. A method of claim 17 wherein said second mammal is subjected to LT-CR for about several weeks longer to about 40 months longer than when said test compound is administered to said first mammal

23. A method of claim 17 wherein said CR is ST-CR wherein said second mammal is subjected to ST-CR for about 1 day to about 8 weeks and wherein said test compound is administered to said first mammal for about 1 day to about 8 weeks.

24. A method of claim 17 wherein said CR is ST-CR wherein said second mammal is subjected to ST-CR for about the same duration of time as said test compound is administered to said first mammal.

25. A method of claim 17 wherein said test compound includes Metformin, Glipizide, Rosiglitazone, Soy Isoflavones, and a combination thereof.

26. A method of claim 17 wherein said test compound being a glucoregulatory agent.

27. A method of claim 17 wherein said changes induced by said CR including comparing changes in gene expression.

28. A method of claim 17 wherein said mammals include mice.

29. A method of claim 17 wherein said changes induced by said CR include at least one of extending life of said mammals that are otherwise healthy and delaying onset of age related diseases.

30. A method of claim 17 wherein said changes induced by said CR include at least one of extending life of mice that are otherwise healthy and delaying onset of age related diseases in mice.

31. A method of claim 17 wherein said changes induced by said CR include changes to genes listed in Tables 3, 4, 5, 6, 7, and 8.

32. A method of reproducing effects of CR comprising:
administering an effective dosage of at least one of Metformin, Glipizide, Rosiglitazone, and Soy Isoflavones to a mammal for an effective amount of time.

33. A method for searching for a compound comprising:
administering a ST-CR diet program to a first group of mammals for a predetermined amount of time;
administering a dosage of at least one compound, for a term which is less than or equal to said predetermined amount of time, to a second group of mammals;
assessing changes in gene expression levels, levels of nucleic acids, proteins, or protein activity levels; and
determining whether said at least one compound mimics at least some effects induced by said ST-CR diet program

34. A method of claim 33 wherein said predetermined amount of time is about eight weeks.

35. A method of claim 33 said compound includes at least one of Metformin, Glipizide, Rosiglitazone, Soy Isoflavones, and a combination thereof.

36. A method of claim 33 said compound being a glucoregulatory agent.

37. A method of claim 33 said first group of mammals and said second group of mammals being similar

38. A method of claim 33 said mammals include mice.

39. A method of claim 33 said effects induced by said ST-CR diet program include at least one of extending life of and delaying onset of age related diseases of said mammals that are otherwise healthy.

40. A method of claim 33 said effects induced by said ST-CR diet program include at least one of extending life of and delaying onset of age related diseases of mice that are otherwise healthy.

41. A method of claim 33 said assessing changes in gene expression levels, levels of nucleic acids, proteins, or protein activity levels includes at least comparing changes in gene expression caused to said first group of mammals by said ST-CR and to said second group of mammals by said at least one compound wherein said changes in gene expression include the genes listed in Tables 3, 4, 5, 6, 7, and 8.
